(19) United States
(12) Patent Application Publication
(10) Pub. No.: US 2023/0274825 A1
(43) Pub. Date: Aug. 31, 2023

(54) INFORMATION PROCESSING APPARATUS, METHOD, AND NON-TRANSITORY COMPUTER READABLE MEDIUM

(71) Applicant: TOYOTA JIDOSHA KABUSHIKI KAISHA, Toyota-shi (JP)

(72) Inventors: Yoshikazu JIKUHARA, Miyoshi-shi (JP); Seii SAI, Yokohama-shi (JP); lbuki SHIMADA, Miyoshi-shi (JP); Takahiro AOKI, Saitama-shi (JP); Keishi KINOSHITA, Setagaya-ku (JP)

(21) Appl. No.: 18/173,168

(22) Filed: Feb. 23, 2023

(30) Foreign Application Priority Data
Feb. 25, 2022 (JP) 2022-028699

(51) Int. Cl. G16H 40/20; G16H 40/67; G08G 1/00; G06F 21/62

(52) U.S. Cl. CPC G16H 40/20; G16H 40/67; G08G 1/202; G06F 21/6245

(57) ABSTRACT

A controller of an information processing apparatus is configured to: acquire, for each of multiple users, information regarding a desired date and desired examination item for a medical checkup; acquire, for each of multiple doctors, information regarding an available date and available examination item for medical checkups; match the multiple users with the multiple doctors, based on the desired date, the desired examination item, the available date, and the available examination item; and determine, when the establishment number of matchings exceeds a predetermined threshold, to perform medical checkups on users related to the matchings the establishment number of which exceeds the predetermined threshold, as patients.

### CROSS-REFERENCE TO RELATED APPLICATION

This application claims priority to Japanese Patent Application No. 2022-028699 filed on Feb. 25, 2022, the entire contents of which are incorporated herein by reference.

### TECHNICAL FIELD

The present disclosure relates to an information processing apparatus, a method, and a program.

### BACKGROUND

Technology for providing services related to medical checkups using vehicles is known. For example, Patent Literature (PTL) 1 discloses technology for setting equipment to be used for medical checkups in vehicles, based on information on users to be diagnosed.

### CITATION LIST

Patent Literature
PTL 1: JP 2021-022332 A

### SUMMARY

When medical checkups are performed by dispatching a vehicle equipped with medical equipment, the dispatch of the vehicle incurs costs, which may be economically unprofitable depending on the number of people who receive the medical checkups. Therefore, there is room for improvement with respect to technology for providing services related to medical checkups using vehicles.

It would be helpful to improve technology for providing services related to medical checkups using vehicles.

An information processing apparatus according to an embodiment of the present disclosure includes a controller configured to:

acquire, for each of multiple users, information regarding a desired date and desired examination item for a medical checkup; acquire, for each of multiple doctors, information regarding an available date and available examination item for medical checkups;

match the multiple users with the multiple doctors, based on the desired date, the desired examination item, the available date, and the available examination item; and

determine, when the establishment number of matchings exceeds a predetermined threshold, to perform medical checkups on users related to the matchings the establishment number of which exceeds the predetermined threshold, as patients.

A method according to an embodiment of the present disclosure is a method performed by an information processing apparatus, the method including:

acquiring, for each of multiple users, information regarding a desired date and desired examination item for a medical checkup;

acquiring, for each of multiple doctors, information regarding an available date and available examination item for medical checkups;

matching the multiple users with the multiple doctors, based on the desired date, the desired examination item, the available date, and the available examination item; and

determining, when the establishment number of matchings exceeds a predetermined threshold, to perform medical checkups on users related to the matchings the establishment number of which exceeds the predetermined threshold, as patients.

A program according to an embodiment of the present disclosure is configured to cause a computer to execute operations, the operations including:

acquiring, for each of multiple users, information regarding a desired date and desired examination item for a medical checkup;

acquiring, for each of multiple doctors, information regarding an available date and available examination item for medical checkups;

matching the multiple users with the multiple doctors, based on the desired date, the desired examination item, the available date, and the available examination item; and

determining, when the establishment number of matchings exceeds a predetermined threshold, to perform medical checkups on users related to the matchings the establishment number of which exceeds the predetermined threshold, as patients.

According to an embodiment of the present disclosure, technology for providing services related to medical checkups using vehicles can be improved.

### BRIEF DESCRIPTION OF THE DRAWINGS

In the accompanying drawings:

FIG. 1 is a block diagram illustrating a schematic configuration of a system according to an embodiment of the present disclosure;

FIG. 2 is a block diagram illustrating a schematic configuration of an information processing apparatus according to the embodiment of the present disclosure;

FIG. 3 is a block diagram illustrating a schematic configuration of a user terminal according to the embodiment of the present disclosure;

FIG. 4 is a block diagram illustrating a schematic configuration of a doctor terminal according to the embodiment of the present disclosure; and

FIG. 5 is a flowchart illustrating operations of the information processing apparatus according to the embodiment of the present disclosure.

### DETAILED DESCRIPTION

Hereinafter, an embodiment of the present disclosure will be described.

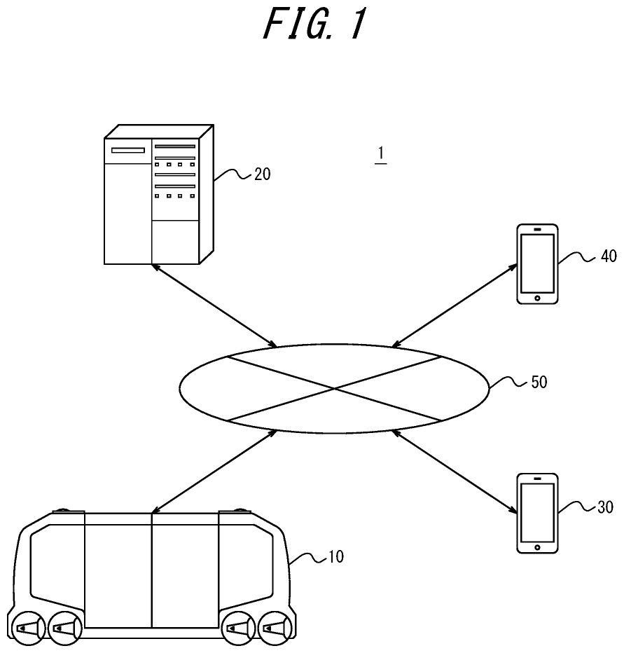

An outline of a system 1 according to the embodiment of the present disclosure will be described with reference to FIG. 1.

The system 1 includes a vehicle 10, an information processing apparatus 20, a user terminal 30, and a doctor terminal 40. The vehicle 10, the information processing apparatus 20, the user terminal 30, and the doctor terminal 40 are communicably connected to a network 50.

The vehicle 10 is an automobile, for example, but is not limited to this, and may be any appropriate vehicle. The automobile is, for example, a gasoline vehicle, a battery electric vehicle (BEV), a hybrid electric vehicle (HEV), a plug-in hybrid electric vehicle (PHEV), a fuel cell electric vehicle (FCEV), or the like, but is not limited to these.

The vehicle 10 may be driven by a driver, or the driving may be automated at any level. The level of automation is, for example, one of level 1 to level 5 according to the classification of the Society of Automotive Engineers (SAE). The vehicle 10 may be a dedicated Mobility as a Service (MaaS) vehicle.

The information processing apparatus 20 is managed by a medical checkup service provider or the like. The information processing apparatus 20 is a computer such as a server that belongs to a cloud computing system or another type of computing system.

The user terminal 30 is used by a user or the like who plans to have a medical checkup. The user terminal 30 is, for example, a mobile device such as a mobile phone, a smartphone, or a tablet.

The doctor terminal 40 is used by a doctor, a nurse, or the like. The doctor terminal 40 is, for example, a mobile device such as a mobile phone, a smartphone, or a tablet.

The network 50 includes the Internet, at least one wide area network (WAN), at least one metropolitan area network (MAN), or any combination thereof. The network 50 may include at least one wireless network, at least one optical network, or any combination thereof. The wireless network is, for example, an ad hoc network, a cellular network, a wireless local area network (LAN), a satellite communication network, or a terrestrial microwave network.

An outline of the present embodiment will be described with reference to FIG. 1.

A controller 21 of the information processing apparatus 20 acquires, for each of multiple users, information regarding a desired date and desired examination item for a medical checkup. The controller 21 also acquires, for each of multiple doctors, information regarding an available date and available examination item for medical checkups. The controller 21 then matches the multiple users with the multiple doctors, based on the desired date, the desired examination item, the available date, and the available examination item. When the establishment number of matchings exceeds a predetermined threshold, the controller 21 determines to perform medical checkups on users related to the matchings the establishment number of which exceeds the predetermined threshold, as patients.

Thus, according to the present embodiment, even though, for example, medical checkups are performed by dispatching the vehicle equipped with medical equipment, the medical checkups are performed only when the establishment number of matchings exceeds the predetermined threshold, as described above, which makes the medical checkups profitable. Thus, technology for providing services related to medical checkups using vehicles can be improved.

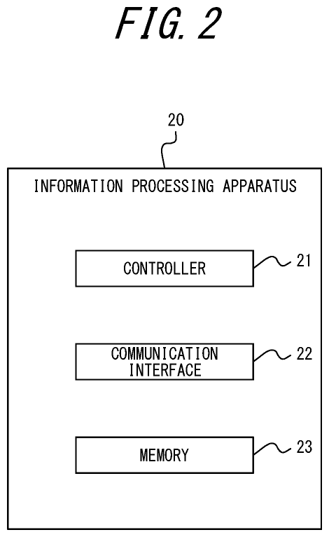

A configuration of the information processing apparatus 20 according to the present embodiment will be described with reference to FIG. 2.

The information processing apparatus 20 includes the controller 21, a communication interface 22, and a memory 23.

The controller 21 includes at least one processor, at least one programmable circuit, at least one dedicated circuit, or any combination thereof. The processor is a general purpose processor such as a central processing unit (CPU) or a graphics processing unit (GPU), or a dedicated processor that is dedicated to specific processing. The programmable circuit is, for example, a field-programmable gate array (FPGA). The dedicated circuit is, for example, an application specific integrated circuit (ASIC). The controller 21 executes processes related to operations of the information processing apparatus 20 while controlling components of the information processing apparatus 20.

The communication interface 22 includes at least one interface for communication. The interface for communication is compliant with, for example, a mobile communication standard, a wired LAN standard, or a wireless LAN standard, but not limited to these, and may be compliant with any communication standard. The communication interface 22 receives data to be used for the operations of the information processing apparatus 20, and transmits data obtained by the operations of the information processing apparatus 20.

The memory 23 includes at least one semiconductor memory, at least one magnetic memory, at least one optical memory, or any combination thereof. The semiconductor memory is, for example, random access memory (RAM) or read only memory (ROM). The RAM is, for example, static random access memory (SRAM) or dynamic random access memory (DRAM). The ROM is, for example, electrically erasable programmable read only memory (EEPROM). The memory 23 functions as, for example, a main memory, an auxiliary memory, or a cache memory. The memory 23 stores data to be used for the operations of the information processing apparatus 20 and data obtained by the operations of the information processing apparatus 20. In the present embodiment, the data to be used for the operations of the information processing apparatus 20 includes a system program, an application program, a database, map information, and the like.

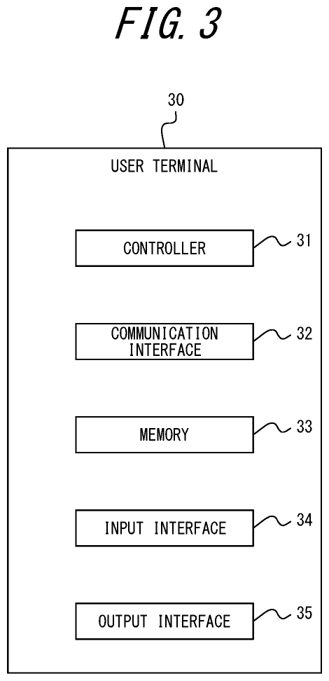

A configuration of the user terminal 30 according to the present embodiment will be described with reference to FIG. 3.

The user terminal 30 includes a controller 31, a communication interface 32, a memory 33, an input interface 34, and an output interface 35.

The controller 31 includes at least one processor, at least one programmable circuit, at least one dedicated circuit, or any combination thereof. The processor is a general purpose processor such as a CPU or a GPU, or a dedicated processor that is dedicated to specific processing. The programmable circuit is, for example, an FPGA. The dedicated circuit is, for example, an ASIC. The controller 31 executes processes related to operations of the user terminal 30 while controlling components of the user terminal 30.

The communication interface 32 includes at least one interface for communication. The interface for communication is, for example, an interface compliant with a mobile communication standard such as a Long Term Evolution (LTE), the 4th generation (4G) standard, or the 5th generation (5G) standard, an interface compliant with a short-range wireless communication standard such as Bluetooth® (Bluetooth is a registered trademark in Japan, other countries, or both), or a LAN interface. The communication interface 32 receives data to be used for the operations of the user terminal 30 and transmits data obtained by the operations of the user terminal 30.

The memory 33 includes at least one semiconductor memory, at least one magnetic memory, at least one optical memory, or any combination thereof. The semiconductor memory is, for example, RAM or ROM. The RAM is, for example, SRAM or DRAM. The ROM is, for example, EEPROM. The memory 33 functions as, for example, a main memory, an auxiliary memory, or a cache memory. The memory 33 stores data to be used for the operations of the user terminal 30 and data obtained by the operations of the user terminal 30.

The input interface 34 includes at least one interface for input. The interface for input is, for example, a physical key, a capacitive key, a pointing device, a touch screen integrally provided with a display, a camera, a light detection and ranging or laser imaging, detection, and ranging (LiDAR) sensor, or a microphone. The input interface 34 accepts an operation for inputting data to be used for the operations of the user terminal 30. The input interface 34, instead of being included in the user terminal 30, may be connected to the user terminal 30 as an external input device. As an interface for connection, for example, an interface compliant with a standard such as Universal Serial Bus (USB), HDMI® (HDMI is a registered trademark in Japan, other countries, or both), or Bluetooth® can be used.

The output interface 35 includes at least one interface for output. The interface for output is, for example, a display or a speaker. The display is, for example, a liquid crystal display (LCD) or an organic electro-luminescent (EL) display. The output interface 35 outputs data obtained by the operations of the user terminal 30. The output interface 35, instead of being included in the user terminal 30, may be connected to the user terminal 30 as an external output device. As an interface for connection, for example, an interface compliant with a standard such as USB, HDMI®, or Bluetooth® can be used.

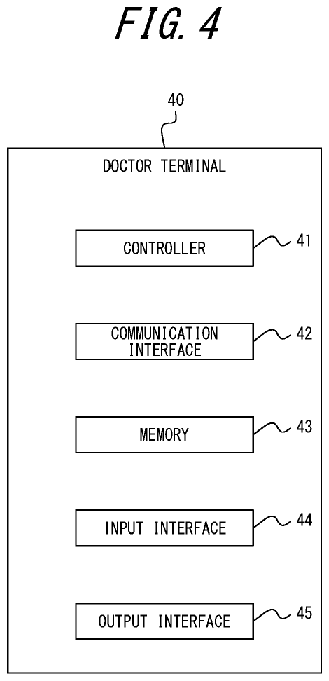

A configuration of the doctor terminal 40 according to the present embodiment will be described with reference to FIG. 4.

The doctor terminal 40 includes a controller 41, a communication interface 42, a memory 43, an input interface 44, and an output interface 45.

The controller 41 includes at least one processor, at least one programmable circuit, at least one dedicated circuit, or any combination thereof. The processor is a general purpose processor such as a CPU or a GPU, or a dedicated processor that is dedicated to specific processing. The programmable circuit is, for example, an FPGA. The dedicated circuit is, for example, an ASIC. The controller 41 executes processes related to operations of the doctor terminal 40 while controlling components of the doctor terminal 40.

The communication interface 42 includes at least one interface for communication. The interface for communication is, for example, an interface compliant with a mobile communication standard such as LTE, the 4G standard, or the 5G standard, an interface compliant with a short-range wireless communication standard such as Bluetooth®, or a LAN interface. The communication interface 42 receives data to be used for the operations of the doctor terminal 40, and transmits data obtained by the operations of the doctor terminal 40.

The memory 43 includes at least one semiconductor memory, at least one magnetic memory, at least one optical memory, or any combination thereof. The semiconductor memory is, for example, RAM or ROM. The RAM is, for example, SRAM or DRAM. The ROM is, for example, EEPROM. The memory 43 functions as, for example, a main memory, an auxiliary memory, or a cache memory. The memory 43 stores data to be used for the operations of the doctor terminal 40 and data obtained by the operations of the doctor terminal 40.

The input interface 44 includes at least one interface for input. The interface for input is, for example, a physical key, a capacitive key, a pointing device, a touch screen integrally provided with a display, a camera, a LiDAR sensor, or a microphone. The input interface 44 accepts an operation for inputting data to be used for the operations of the doctor terminal 40. The input interface 44, instead of being included in the doctor terminal 40, may be connected to the doctor terminal 40 as an external input device. As an interface for connection, for example, an interface compliant with a standard such as USB, HDMI®, or Bluetooth® can be used.

The output interface 45 includes at least one interface for output. The interface for output is, for example, a display or a speaker. The display is, for example, an LCD or an organic EL display. The output interface 45 outputs data obtained by the operations of the doctor terminal 40. The output interface 45, instead of being included in the doctor terminal 40, may be connected to the doctor terminal 40 as an external output device. As an interface for connection, for example, an interface compliant with a standard such as USB, HDMI®, or Bluetooth® can be used.

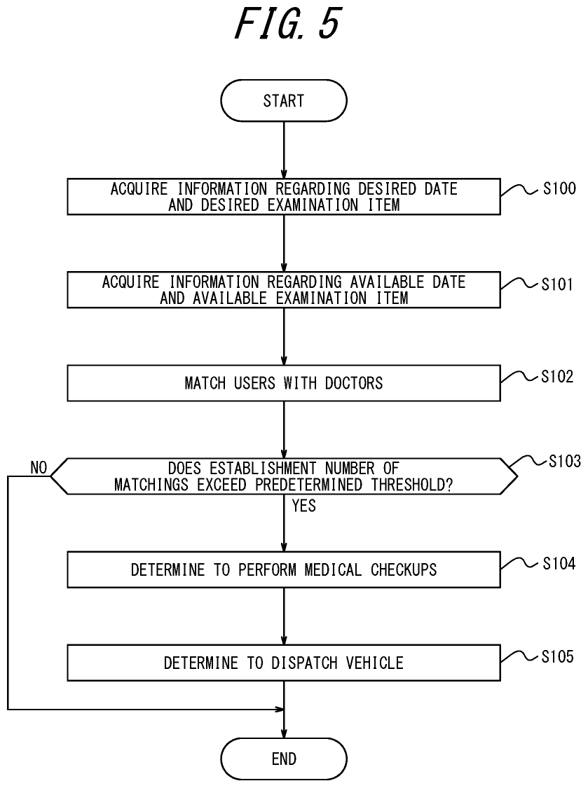

The operations of the information processing apparatus 20 according to the present embodiment will be described with reference to FIG. 5. These operations correspond to a method according to an embodiment of the present disclosure.

Step S100: The controller 21 of the information processing apparatus 20 acquires, for each of multiple users, information regarding a desired date and desired examination item for a medical checkup.

Specifically, the controller 31 of the user terminal 30 controls the input interface 34 to accept operations by each user entering information regarding a desired date and desired examination item for a medical checkup. The operations may be operations on an application regarding medical checkups running on the user terminal 30, but the present disclosure is not limited to these. The controller 31 of the user terminal 30 controls the communication interface 32 to transmit, to the information processing apparatus 20, the information regarding the desired date and desired examination item for the medical checkup. At this time, the controller 31 of the user terminal 30 controls the communication interface 32 to transmit, to the information processing apparatus 20, user information including, for example, a terminal ID of the user terminal 30, location information such as the user's home or workplace, the user's name, and the like. The controller 21 of the information processing apparatus 20 then controls the communication interface 22 to receive, from the user terminal 30, the information regarding the desired date and desired examination item for the medical checkup. The controller 21 of the information processing apparatus 20 then stores the information regarding the desired date and desired examination item for the medical checkup in the memory 23. The information regarding the desired date and desired examination item for the medical checkup and the user information are stored in the memory 23 in association with each other. Here, the desired date for the medical checkup may be selected from multiple candidate dates (or multiple candidate time periods) predetermined by a medical checkup service provider or the like, but the present disclosure is not limited to this. The information regarding the desired examination item may include an examination item for a general medical checkup based on laws and regulations or the like, or an examination item for a special medical checkup based on laws and regulations or the like, but the present disclosure is not limited to these.

Here, the controller 21 of the information processing apparatus may complete the processing of step S100 a predetermined number of days prior to a performance date, which is described later in step S104, in a case in which the performance date is predictable to some extent such that, for example, the desired date for the medical checkup is selected from the multiple candidate dates (or multiple time periods) predetermined by the medical checkup service provider or the like. This allows the medical checkup service provider or the like to know the user's demands for the medical checkup in advance.

Step S101: the controller 21 of the information processing apparatus 20 acquires, for each of doctors, information regarding an available date and available examination item for medical checkups.

Specifically, the controller 41 of the doctor terminal 40 controls the input interface 44 to accept operations by each doctor entering information regarding an available date and available examination item for medical checkups. The operations may be operations on an application related to medical checkups running on the doctor terminal 40, but the present disclosure is not limited to these. The controller 41 of the doctor terminal 40 controls the communication interface 42 to transmit, to the information processing apparatus 20, the information regarding the available date and available examination item for the medical checkups. At this time, the controller 41 of the doctor terminal controls the communication interface 42 to transmit, to the information processing apparatus 20, doctor information including, for example, a terminal ID of the doctor terminal 40, location information such as the doctor's home or workplace, the doctor's name, the doctor's specialty, and the like. The controller 21 of the information processing apparatus 20 then controls the communication interface 22 to receive, from the doctor terminal 40, the information regarding the available date and available examination item for the medical checkups. The controller 21 of the information processing apparatus 20 then stores the information regarding the available date and available examination item for the medical checkups in the memory 23. The information regarding the available date and available examination item for the medical checkups and the doctor information are stored in the memory 23 in association with each other. Here, the available date for the medical checkups may be selected from multiple candidate dates (or multiple candidate time periods) predetermined by the medical checkup service provider or the like, but the present disclosure is not limited to this. The available examination item may be appropriately determined or selected according to each doctor's specialty, may include an examination item for general medical checkups based on laws and regulations or the like, or may include an examination item for special medical checkups based on laws and regulations or the like, but the present disclosure is not limited to these.

Here, in an application related to schedule management running on the doctor terminal 40, the available date of step S101 may be blocked (temporarily scheduled) in the doctor's schedule, until notification, which is described later in step S104, is completed. This allows the medical checkup service provider or the like to temporarily hold the doctors who are available for the medical checkups, thereby preventing a situation in which there is a shortage of doctors for the medical checkups.

Step S102: The controller 21 of the information processing apparatus 20 matches the multiple users with the multiple doctors, based on the desired date of step S100 and the desired examination item of step S100, and the available date of step S101 and the available examination item of step S101.

Specifically, the controller 21 of the information processing apparatus 20 refers to the memory 23 and identifies, for each doctor, users whose desired dates of step S100 are included in the available date of step S101, and whose desired examination items of step S100 are included in the available examination item of step S101. The controller 21 of the information processing apparatus 20 then stores, in the memory 23, the number of the users identified for each doctor, as the establishment number of matchings of users with each doctor. Assuming that, for example, for Doctor A, the number of users whose desired dates are included in the above available date and whose desired examination items are included in the above available examination item is 100. Assuming that, for Doctor B, the number of users whose desired dates are included in the above available date and whose desired examination items are included in the above available examination item is 10. In this case, the establishment number of matchings of users with Doctor A is 100, and the establishment number of matchings of users with Doctor B is 10. However, in the present disclosure, a method of matching is not limited to the method described above, and any matching algorithm can be used. Information indicating users who are matched with each doctor is stored in the memory 23, in association with the user information and doctor information stored in the memory 23.

Step S103: The controller 21 of the information processing apparatus 20 determines whether the establishment number of matchings of step S102 exceeds a predetermined threshold. When it is determined that the establishment number exceeds the predetermined threshold, the process proceeds to step S104. On the other hand, when it is not determined that the establishment number exceeds the predetermined threshold, the process proceeds to step S106.

Here, the predetermined threshold (for example, a minimum number of users to perform medical checkups) may be predetermined by the medical checkup service provider or the like. In this case, costs incurred by dispatching the vehicle 10, which will be described later in step S105, and profits affected by the number of patients of medical checkups could be taken into account.

Step S104: The controller 21 of the information processing apparatus 20 determines to perform medical checkups on users related to matchings the establishment number of which exceeds the predetermined threshold, as patients.

Specifically, the controller 21 of the information processing apparatus 20 generates any message to notify a performance date and performance examination item for a medical checkup. Then, the controller 21 of the information processing apparatus 20 refers to the memory 23 and identifies the user terminals 30 that are in the possession or the like of the users related to the matchings the establishment number of which exceeds the predetermined threshold. The controller 21 of the information processing apparatus 20 also refers to the memory 23 and identifies the doctor terminal 40 that is in the possession or the like of a doctor related to the matchings the establishment number of which exceeds the predetermined threshold. The controller 21 of the information processing apparatus 20 then controls the communication interface 22 to transmit the message to the identified user terminals 30 and the identified doctor terminal 40. The controller 31 of each user terminal 30 controls the communication interface 32 to receive the message from the information processing apparatus 20. The controller 31 of each user terminal 30 then controls the output interface 35 to notify the user of the message through screen display or audio. On the other hand, the controller 41 of the doctor terminal 40 controls the communication interface 42 to receive the message from the information processing apparatus 20. The controller 41 of the doctor terminal 40 then controls the output interface 45 to notify the doctor of the message through screen display or audio. In the specific example described in step S102, the establishment number of matchings with Doctor A is 100, and the establishment number of matchings with Doctor B is 10. Assuming that the predetermined threshold is 50, users who are matched with Doctor A correspond to the “users related to the matchings the establishment number of which exceeds the predetermined threshold” described above. The desired date inputted in step S100 by each of the users who are matched with Doctor A corresponds to the “performance date for a medical checkup” described above. The desired examination item inputted in step S100 by each of the users who are matched with Doctor A corresponds to the “performance examination item for a medical checkup” described above.

Step S105: The controller 21 of the information processing apparatus 20 determines to dispatch the vehicle 10 equipped with the medical equipment to a designated parking space on the performance date for the medical checkups.

Specifically, the controller 21 of the information processing apparatus 20 generates information indicating a driving route of the vehicle 10 equipped with the medical equipment, based on the location information indicating the home, workplace, or the like of each of the users related to the matchings the establishment number of which exceeds the predetermined threshold stored in the memory 23, the location information indicating the home, workplace, or the like of the doctor related to the matchings the establishment number of which exceeds the predetermined threshold stored in the memory 23, and location information indicating the designated parking space predetermined by the medical checkup service provider or the like. Here, assuming that the vehicle 10 equipped with the medical equipment is capable of unmanned automatic driving (for example, automatic driving at level 5 as defined in SAE), for example. The controller 21 of the information processing apparatus 20 then controls the communication interface 22 to transmit the information indicating the driving route to the vehicle 10 equipped with the medical equipment, a predetermined time before a start time of the medical checkups. The vehicle 10 equipped with the medical equipment thereby achieves automatic driving along the driving route, by working in cooperation with one or more sensor modules, including, for example, a velocity sensor, an acceleration sensor, an angular velocity sensor, a LiDAR sensor, a steering wheel angle sensor, and the like. The vehicle equipped with the medical equipment then arrives at the designated parking space a predetermined time before the start time of the medical checkups. The medical checkups are then performed in the vehicle 10 equipped with the medical equipment parked in the designated parking space. In the present disclosure, a location in which the medical checkups are performed is not limited to the above-described parking space, but can be arbitrarily determined by the medical checkup service provide or the like.

As described above, the controller 21 of the information processing apparatus 20 according to the present embodiment acquires, for each of multiple users, information regarding a desired date and desired examination item for a medical checkup. The controller 21 then acquires, for each of multiple doctors, information regarding an available date and available examination item for medical checkups. The controller 21 then matches the multiple users with the multiple doctors, based on the desired date, the desired examination item, the available date, and the available examination item. When the establishment number of matchings exceeds a predetermined threshold, the controller 21 determines to perform medical checkups on users related to the matchings the establishment number of which exceeds the predetermined threshold, as patients.

According to this configuration, even though medical checkups are performed by dispatching the vehicle 10 equipped with the medical equipment, the medical checkups are performed only when the establishment number of matchings exceeds a predetermined threshold, as described above, which makes the medical checkups profitable. Thus, technology for providing services related to medical checkups using vehicles can be improved.

While the present disclosure has been described with reference to the drawings and examples, it should be noted that various modifications and revisions may be implemented by those skilled in the art based on the present disclosure. Accordingly, such modifications and revisions are included within the scope of the present disclosure. For example, functions or the like included in each component, each step, or the like can be rearranged without logical inconsistency, and multiple components, steps, or the like can be combined into one or divided.

As a variation, an embodiment in which the configuration and operations of the information processing apparatus 20 are distributed to multiple computers capable of communicating with each other can be implemented. For example, an embodiment in which some or all of the components of the information processing apparatus 20 are provided in the vehicle 10 can also be implemented.

As another variation, the controller 21 of the information processing apparatus 20 may acquire, for each of multiple nurses, information regarding an available date for medical checkups. The controller 21 of the information processing apparatus 20 may then take into account the acquired available date of each of the multiple nurses in the matching in step S102 described above. Specifically, the controller 21 of the information processing apparatus 20 controls the communication interface 22 to acquire information regarding an available date for medical checkups from any terminal that is in the possession or the like of each nurse, in the same manner as in step S101 described above. The controller 21 of the information processing apparatus 20 then determines, for each of the users who are matched with the doctor in step S102 described above, whether a nurse who is available on the desired date of step S100 is present, based on the acquired information regarding the available date. The controller 21 of the information processing apparatus 20 then identifies users for which nurses are determined to be present, of the users who are matched with the doctor in step S102 described above. The controller 21 of the information processing apparatus 20 then stores the number of the identified users in the memory 23, as the establishment number of matchings. Other processes are the same as those in the embodiment described above, and explanation in the embodiment is applied.

As yet another variation, the controller 21 of the information processing apparatus 20 may acquire information regarding available medical equipment for medical checkups. The controller 21 of the information processing apparatus 20 may then take into account the acquired information regarding the medical equipment in the matching in step S102 described above. Specifically, the memory 23 of the information processing apparatus 20 stores in advance, for example, information indicating one or more types of medical equipment capable of being equipped in the vehicle 10 and available time periods thereof. The controller 21 of the information processing apparatus 20 then refers to the memory 23 and determines, for each of the users who are matched with the doctor in step S102 described above, whether medical equipment that is available on the desired date of step S100 for the examination item of step S100 is present, based on the acquired types of medical equipment and available time periods. The controller 21 of the information processing apparatus 20 then identifies users for which the medical equipment is determined to be present, of the users who are matched with the doctor in step S102 described above. The controller 21 of the information processing apparatus 20 then stores the number of the identified users in the memory 23, as the establishment number of matchings. Other processes are the same as those in the embodiment described above, and explanation in the embodiment is applied.

As yet another variation, the controller 21 of the information processing apparatus 20 may, similarly to step S102, re-match a user with respect to whom the establishment number is not determined to exceed the predetermined threshold in step S103 described above, based on a candidate date other than the performance date for the medical checkups of step S104, of the multiple candidate dates (or multiple candidate time periods) included in the desired date acquired in step S100 described above. Other processes are the same as those in the embodiment described above, and explanation in the embodiment is applied.

As yet another variation, the controller 21 of the information processing apparatus 20 may perform the following process for each of the patients determined in step S104 described above, based on information regarding a progress status of the medical checkup. In other words, the controller 21 of the information processing apparatus may determine, for each of the patients, whether an uncompleted examination item is to occur or whether an uncompleted examination item has occurred, based on information regarding a progress status of the medical checkup. When it is determined that the uncompleted examination item is to occur or has occurred, the controller 21 of the information processing apparatus 20 may acquire information regarding another desired date, which is different from the desired date acquired in step S100 described above, for the uncompleted examination item. Then, the controller 21 of the information processing apparatus 20 may re-match the patient as in step S102 described above, based on the acquired desired date for the uncompleted examination item. Specifically, the controller 21 of the information processing apparatus 20 may determine that one or more uncompleted examination items may occur or have occurred, from a ratio of the number of examination items that have already been performed in the medical checkup to the number of all examination items planned to be performed in the medical checkup. More specifically, the controller 21 of the information processing apparatus determines that one or more uncompleted examination items may occur when the ratio at a given time on the performance date for the medical checkup is less than a predetermined threshold. On the other hand, the controller 21 of the information processing apparatus 20 determines that one or more uncompleted examination items have occurred when the ratio is less than 100% after the completion of the medical checkup. The controller 21 of the information processing apparatus 20 may control the communication interface 22 to appropriately acquire the ratio from the user terminal 30 or the doctor terminal 40. The controller 21 of the information processing apparatus then controls the communication interface 22 to appropriately acquire, from the user terminal 30, the user's response to a question asking for another desired date for the uncompleted examination items, different from the desired date acquired in step S100. Then, the controller 21 of the information processing apparatus 20 matches, by the same method as in the above step S102, a patient with respect to whom it is determined that an uncompleted examination item is likely to occur or has occurred, as a target of matching, using the other desired date included in the response. Other processes are the same as those in the embodiment described above, and explanation in the embodiment is applied.

As yet another variation, in a case in which information regarding a medical history or disability of each of the multiple users is acquired before performing the medical checkup, the controller 21 of the information processing apparatus 20 may register the acquired information regarding the medical history or disability as secret data, at least until it is determined, by the above-described matching, to perform the medical checkup. Specifically, the secret data is, for example, data that is stored in the memory 23 of the information processing apparatus 20 and is prohibited by any encryption algorithm from being viewed by third parties who do not have access to the information processing apparatus 20. The third parties may be persons other than the doctors, or persons other than the doctor related to the matchings the establishment number of which exceeds the predetermined threshold as described above.

As yet another variation, an embodiment in which a general purpose computer functions as the information processing apparatus according to the above embodiment can also be implemented. Specifically, a program in which processes for realizing the functions of the information processing apparatus 20 according to the above embodiment are written may be stored in a memory of a general purpose computer, and the program may be read and executed by a processor. Accordingly, the present disclosure can also be implemented as a program executable by a processor, or a non-transitory computer readable medium storing the program.

1. An information processing apparatus comprising a controller configured to:
acquire, for each of multiple users, information regarding a desired date and desired examination item for a medical checkup;
acquire, for each of multiple doctors, information regarding an available date and available examination item for medical checkups;
match the multiple users with the multiple doctors, based on the desired date, the desired examination item, the available date, and the available examination item; and
determine, when an establishment number of matchings exceeds a predetermined threshold, to perform medical checkups on users related to the matchings the establishment number of which exceeds the predetermined threshold, as patients.

2. The information processing apparatus according to claim 1, wherein the controller is configured to determine to dispatch a vehicle equipped with medical equipment to a designated parking space on a performance date for the medical checkups on the patients.

3. The information processing apparatus according to claim 1, wherein the controller is configured to:
acquire, for each of multiple nurses, information regarding an available date for medical checkups; and
take into account the available date of each of the multiple nurses in matching the multiple users with the multiple doctors.

4. The information processing apparatus according to claim 1, wherein the controller is configured to:
acquire information regarding medical equipment available for medical checkups; and
take into account the information regarding the medical equipment in matching the multiple users with the multiple doctors.

5. The information processing apparatus according to claim 1, wherein
the desired date includes multiple candidate dates, and
the controller is configured to re-match a user, among the multiple users, with respect to whom the establishment number is not determined to exceed the predetermined threshold with the multiple doctors, based on a candidate date, among the multiple candidate dates, other than a performance date for the medical checkups on the patients.

6. The information processing apparatus according to claim 1, wherein the controller is configured to:
determine, for each of the patients, whether an uncompleted examination item is to occur or whether an uncompleted examination item has occurred, based on information regarding a progress status of a corresponding medical checkup; and
upon determining that the uncompleted examination item is to occur or has occurred, re-match a user, among the multiple users, with respect to whom it is determined that the uncompleted examination item is to occur or has occurred with the multiple doctors, for the uncompleted examination item, based on another desired date different from the desired date.

7. The information processing apparatus according to claim 1, when information regarding a medical history or disability of each of the multiple users is acquired before performing the medical checkups on the patients, the controller registers the acquired information regarding the medical history or disability as secret data, at least until it is determined to perform the medical checkups on the patients.

8. A method performed by an information processing apparatus, the method comprising:
acquiring, for each of multiple users, information regarding a desired date and desired examination item for a medical checkup;
acquiring, for each of multiple doctors, information regarding an available date and available examination item for medical checkups;
matching the multiple users with the multiple doctors, based on the desired date, the desired examination item, the available date, and the available examination item; and
determining, when an establishment number of matchings exceeds a predetermined threshold, to perform medical checkups on users related to the matchings the establishment number of which exceeds the predetermined threshold, as patients.

9. The method according to claim 8, further comprising determining to dispatch a vehicle equipped with medical equipment to a designated parking space on a performance date for the medical checkups on the patients.

10. The method according to claim 8, further comprising:
acquiring, for each of multiple nurses, information regarding an available date for medical checkups; and
taking into account the available date of each of the multiple nurses in matching the multiple users with the multiple doctors.

11. The method according to claim 8, further comprising:
acquiring information regarding medical equipment available for medical checkups; and
taking into account the information regarding the medical equipment in matching the multiple users with the multiple doctors.

12. The method according to claim 8, wherein
the desired date includes multiple candidate dates, and
the method further comprises re-matching a user, among the multiple users, with respect to whom the establishment number is not determined to exceed the predetermined threshold with the multiple doctors, based on a candidate date, among the multiple candidate dates, other than a performance date for the medical checkups on the patients.

13. The method according to claim 8, further comprising:
determining, for each of the patients, whether an uncompleted examination item is to occur or whether an uncompleted examination item has occurred, based on information regarding a progress status of a corresponding medical checkup; and
upon determining that the uncompleted examination item is to occur or has occurred, re-matching a user, among the multiple users, with respect to whom it is determined that the uncompleted examination item is to occur or has occurred with the multiple doctors, for the uncompleted examination item, based on another desired date different from the desired date.

14. The method according to claim 8, further comprising, when information regarding a medical history or disability of each of the multiple users is acquired before performing the medical checkups on the patients, registering the acquired information regarding the medical history or disability as secret data, at least until it is determined to perform the medical checkups on the patients.

15. A non-transitory computer readable medium storing a program configured to cause a computer to execute operations, the operations comprising:
acquiring, for each of multiple users, information regarding a desired date and desired examination item for a medical checkup;
acquiring, for each of multiple doctors, information regarding an available date and available examination item for medical checkups;
matching the multiple users with the multiple doctors, based on the desired date, the desired examination item, the available date, and the available examination item; and
determining, when an establishment number of matchings exceeds a predetermined threshold, to perform medical checkups on users related to the matchings the establishment number of which exceeds the predetermined threshold, as patients.

16. The non-transitory computer readable medium according to claim 15, wherein the operations further comprise determining to dispatch a vehicle equipped with medical equipment to a designated parking space on a performance date for the medical checkups on the patients.

17. The non-transitory computer readable medium according to claim 15, wherein the operations further comprise:
acquiring, for each of multiple nurses, information regarding an available date for medical checkups; and
taking into account the available date of each of the multiple nurses in matching the multiple users with the multiple doctors.

18. The non-transitory computer readable medium according to claim 15, wherein the operations further comprise:
acquiring information regarding medical equipment available for medical checkups; and
taking into account the information regarding the medical equipment in matching the multiple users with the multiple doctors.

19. The non-transitory computer readable medium according to claim 15, wherein
the desired date includes multiple candidate dates, and
the operations further comprise re-matching a user, among the multiple users, with respect to whom the establishment number of which is not determined to exceed the predetermined threshold with the multiple doctors, based on a candidate date, among the multiple candidate dates, other than a performance date for the medical checkups on the patients.

20. The non-transitory computer readable medium according to claim 15, wherein the operations further comprise:
determining, for each of the patients, whether an uncompleted examination item is to occur or whether an uncompleted examination item has occurred, based on information regarding a progress status of a corresponding medical checkup; and
upon determining that the uncompleted examination item is to occur or has occurred, re-matching a user, among the multiple users, with respect to whom it is determined that the uncompleted examination item is to occur or has occurred with the multiple doctors, for the uncompleted examination item, based on another desired date different from the desired date.
